(19) United States
(12) Patent Application Publication
(10) Pub. No.: US 2019/0032106 A1
(43) Pub. Date: Jan. 31, 2019

(54) PREIMPLANTATION SCREENING

(71) Applicant: University of Warwick, United Kingdom (GB)

(72) Inventors: Scarlett SALTER, Frome, Somerset (GB); Jan BROSENS, Leamington Spa, Warwickshire (GB)

(21) Appl. No.: 15/775,223

(22) PCT Filed: Nov. 11, 2016
(86) PCT No.: PCT/GB2016/053535

§ 371 Date: May 10, 2018

(30) Foreign Application Priority Data
Nov. 12, 2015 (GB) 1519944.1

(51) Int. Cl. C12Q 1/37; G01N 33/68

(52) U.S. Cl. CPC C12Q 1/37; C12M 41/46; G01N 33/689

(57) ABSTRACT

The present invention relates to a method of assessing the viability of an embryo, wherein the method comprises a step of: measuring the level and/or activity of a protein marker selected from either TMPRSS2 or PRSS8 in a sample of culture medium, wherein the sample has been obtained from the in vitro culture medium of an embryo produced by in vitro fertilization, wherein the level and/or activity of the protein marker is indicative of the viability of the embryo.

### CROSS REFERENCE TO RELATED APPLICATIONS

This application is a U.S. National Phase application of International Appl. No. PCT/GB2016/053535, filed Nov. 11, 2016, which claims priority to United Kingdom Appl. No. GB 1519944.1, filed Nov. 12, 2015, each of which is incorporated herein by reference.

### FIELD OF THE INVENTION

The present invention relates to methods of assessing the viability of embryos following in vitro fertilization (IVF). The embryos are assessed for the purposes of assessing their suitability for embryo transfer to a female recipient. The invention also relates to methods of selecting embryos suitable for implantation based on a comparison of embryos produced by IVF carried out with oocytes from the same female donor. The methods of the invention are based on measuring the levels and/or activity of a protein marker, selected from either TMPRSS2 or PRSS8, in samples of embryo-conditioned culture media. The levels of these two markers in embryo-conditioned media have been found to correlate well with the viability of embryos post-implantation, rendering these markers particularly suitable for use in the screening methods of the invention.

### BACKGROUND TO THE INVENTION

In vitro fertilization (IVF) has a limited success rate with less than 40% of IVF treatments resulting in live births (www.nhs.uk/conditions/IVF). A major contributing factor to the low success rate is the difficulty in selecting good quality embryos for uterine implantation, particularly in cases of ‘single embryo transfer’ where only one embryo is transferred to a mother to avoid problems with multiple births. Human embryos are morphologically and chromosomally diverse with at least two-thirds of human pre-implantation embryos containing chromosomal errors or aneuploid cells, which may impede normal embryo development. Defective embryos of this nature are either unsuitable for implantation altogether, or in some cases will result in implantation followed by miscarriage of the unviable or “developmentally-impaired” embryo. The development of a reliable method for assessing human embryo viability remains a significant challenge for improving pregnancy rates in IVF clinics.

Techniques exist for screening embryos generated by IVF prior to implantation to assess the quality of the embryo. These techniques vary in their accuracy, ease-of-use and invasiveness. Current techniques include morphological screening, time-lapse imaging and pre-implantation genetic screening (PGS). However, there are disadvantages associated with these existing techniques (see review by Bolton et al. 2015 Human Fertility 18:3, 156-164).

Morphological screening involves microscopic analysis of embryos at a particular stage of development, or throughout development in the case of time-lapse imaging. The embryos are then graded by an embryologist according to certain morphological criteria such as stage of development, cell size, cell shape, granularity, apoptosis and expansion. There are numerous grading schemes and methodologies and thus classification is somewhat variable and subjective between centres. Moreover, morphological examination is not always a reliable means by which to detect those chromosomal abnormalities that occur in unviable embryos. As such, the transfer of embryos deemed of “top-quality” via this method only results in pregnancy in 36.5% of cases (De Neubourg et al. 2004 Human Reproduction 19:6, 1476-1479).

PGS (Pre-implantation genetic screening) is the genetic analysis of the chromosome status of one or two cells removed from the embryo via aspiration. Typically this involves the application of array comparative genomic hybridisation (aCGH) or next-generation sequencing (NGS) on trophectoderm cells removed from a blastocyst. While this technique allows for the more detailed analysis of potential genetic abnormalities, PGS requires cells to be biopsied from the developing embryo and is therefore an invasive technique that is not free of risk. The embryo is mechanically removed from its outer shell so that cells can be aspirated, which may result in physical damage to the embryo at the time of biopsy. The full consequences of this procedure, particularly the long-term effects, are incompletely understood.

PGS is further complicated by a phenomenon known as mosaicism, whereby the biopsied single cell is not representative of the chromosomal status of the embryo. For instance, discordance in the ploidy status between the inner cell mass and the cells of the external trophectoderm is relatively common (Brezina & Kutteh, 2015 BMJ 350, 7611), which can result in misdiagnosis. An additional limitation of PGS is that the procedure requires at least two top quality blastocysts for analysis and is thus inapplicable to many patients.

More recently, researchers have looked to the possibility of analysing markers in the culture medium of IVF embryos (known as the embryonic secretome) as a means to identify viable embryos for implantation. Previous research suggests that perturbations in metabolism may be a reflection of the embryo's inherent viability (Gardner et al. 2011 Human Reproduction 26:8, 1981-6; Guerif et al. 2013 PLoS ONE 8(8): e67834). Embryo-conditioned medium (ECM) may be analysed for metabolic markers such as glucose, lactate and oxygen consumption or amino acid utilisation, in order to predict embryo viability. However, the requirement for complex technical equipment such as near-infrared spectroscopy or nuclear magnetic resonance spectroscopy makes measuring metabolites difficult to implement in routine clinical practices.

Studies analysing embryonic protein secretome profiles found that it was possible to discriminate between euploid and aneuploid status. In particular, lipocalin-1 was identified as a marker of aneuploidy, and was significantly upregulated in the culture media from aneuploid blastocysts compared to media from euploid blastocysts (McReynolds et al. 2011 American Society for Reproductive Medicine 95:8, 2631-2633).

What these existing techniques fail to address is that successful implantation is dependent on a combination of factors that includes chromosome ploidy status but also includes the expression of other presently unknown implantation factors. For instance, an identified supposedly ‘healthy’ euploid embryo could still have poor implantation potential. Thus, there is a need to find other markers indicative of successful implantation for use in assessing pre-implantation embryo viability. Furthermore, many of the existing techniques are directed towards identifying developmentally-impaired embryos (DIE) to ‘de-select’ before implantation, rather than identifying embryos with the greatest potential for successful implantation.

One recent study (Brosens et al. 2014 Scientific Reports 4: 3894) concerned with implantation factors describes the potential signals communicated from the implanted embryo to the endometrial epithelial cells (EEC) of the uterus lining. The researchers found that the maternal endometrial cells responded differently when exposed to media from “developmentally-competent embryos” (DCE) or to media from “developmentally-impaired embryos” (DIE). More specifically, soluble factors secreted by DIEs perturbed multiple genes in the maternal endometrial cells relating to the endoplasmic reticulum stress response pathway. In contrast, soluble factors secreted by DCEs affected a number of uterine genes responsible for promoting implantation. Two implantation-specific proteases, which exhibit trypsin-like activity, were found to be strongly induced in mouse uterus by signals from DCEs. However, no homologs were found in humans.

Despite extensive research into methods of assessing embryo viability pre-implantation for embryos produced by IVF, the existing methods are associated with significantly high levels of false positives and negative misdiagnoses. In light of the above, there is a need to improve techniques and develop more reliable selection methods for identifying viable embryos with the greatest potential to implant and lead to a successful pregnancy. The accurate selection of a viable embryo for single embryo transfer or for cryopreservation is critical to improving the overall success rates of IVF treatment. The present invention fulfils this need.

### SUMMARY OF INVENTION

The present inventors have identified two proteins, TMPRSS2 and PRSS8, in the culture media of IVF embryos, the levels of which can be used to predict embryo viability. The expression of both TMPRSS2 and PRSS8 is upregulated as the embryo progresses to the blastocyst stage. Importantly, these markers have been found to correlate with embryo viability as assessed by successful embryo implantation potential. The invention is based on the surprising discovery that the conditioned media from viable embryos typically contains lower levels of TMPRSS2 and higher levels of PRSS8 than the conditioned media from non-viable embryos. It follows, that measuring the levels of these markers in embryo-conditioned media can be used as an effective means to assess embryo viability.

In a first aspect, the present invention provides a method of assessing the viability of an embryo, wherein the method comprises a step of:
measuring the level and/or activity of a protein marker selected from either TMPRSS2 or PRSS8 in a sample of culture medium, wherein the sample has been obtained from the in vitro culture medium of an embryo produced by in vitro fertilization,
wherein the level and/or activity of the protein marker is indicative of the viability of the embryo.

In certain embodiments, the viability of the embryo is assessed by determining the level and/or activity of the protein marker relative to a sample of control unconditioned culture medium, a “control”. For such embodiments wherein the protein marker is TMPRSS2, if the level and/or activity of TMPRSS2 is increased by less than 40% relative to control, the embryo is assessed as viable and if the level and/or activity of TMPRSS2 is increased by more than 40% relative to control, the embryo is not viable. For such embodiments wherein the protein marker is PRSS8, if the level and/or activity of PRSS8 is increased relative to control, the embryo is assessed as viable, and if the level and/or activity of PRSS8 is equivalent to or decreased relative to control, the embryo is not viable.

In certain embodiments, the methods may comprise an additional step of:
(ii) comparing the level and/or activity of the protein marker measured in the sample with a reference value for said protein marker, wherein the level and/or activity of the protein marker relative to the reference value is indicative of the viability of the embryo. For such embodiments wherein the protein marker is TMPRSS2, the reference value may be indicative of a viable embryo such that if the level and/or activity of TMPRSS2 is equal to or lower than the reference value the embryo is assessed as viable, and if the level and/or activity of TMPRSS2 is higher than the reference value the embryo is assessed as not viable. Alternatively, the reference value may be indicative of a non-viable embryo such that if the level and/or activity of TMPRSS2 is lower than the reference value the embryo is assessed as viable, and if the level and/or activity of TMPRSS2 is equal to or higher than the reference value the embryo is assessed as not viable. For such embodiments wherein the protein marker is PRSS8, the reference value may be indicative of a viable embryo such that if the level and/or activity of PRSS8 is equal to or higher than the reference value the embryo is assessed as viable, and if the level and/or activity of PRSS8 is lower than the reference value the embryo is assessed as not viable. Alternatively, the reference value may be indicative of a non-viable embryo such that if the level of PRSS8 is higher than the reference value the embryo is assessed as viable, and if the level of PRSS8 is equal to or lower than the reference value the embryo is assessed as not viable.

In a second aspect, the present invention provides a method of selecting an embryo suitable for implantation, the method comprising the steps of:
(i) measuring the level and/or activity of a protein marker selected from either TMPRSS2 or PRSS8 in two or more samples of culture medium, wherein the samples have been obtained from the in vitro culture media of embryos produced by in vitro fertilization using oocytes from the same female donor,
(ii) comparing the level and/or activity of the protein marker between the two or more samples of (i),
   wherein if the marker is TMPRSS2, the embryo from which the sample of culture medium having the lowest TMPRSS2 level and/or activity is obtained is selected for implantation, or, if the marker is PRSS8, the embryo from which the sample of culture medium having the highest PRSS8 level and/or activity is obtained is selected for implantation.

In a third aspect, the present invention provides use of a protein marker selected from TMPRSS2 and PRSS8 to assess the viability of an embryo produced by in vitro fertilization.

In a fourth aspect, the present invention provides use of a protein marker selected from TMPRSS2 and PRSS8 to select an embryo produced by in vitro fertilization for implantation.

In all aspects of the invention, in preferred embodiments, the embryo is a human embryo.

### BRIEF DESCRIPTION OF THE DRAWINGS

FIG. 1 depicts the results of in silico analysis of publicly-available datasets from the Gene Expression Omnibus (Edgar et al. 2002 Nucleic Acids Research, 30:1, 207-210), accession number GSD3959 (homo sapiens) and GSD813 (mus musculus). The analysis was carried out to identify serine protease genes significantly upregulated at the blastocyst stage in humans and mice. Out of 194 human and 228 mouse serine protease genes, only 2 were conserved. These were TMPRSS2 and PRSS8. SPSS Software (IBM) was used for statistical analysis.

FIG. 2 shows the results of experiments carried out to assess the protease activity (A) and trypsin activity (B) in embryo-conditioned media harvested on day 2 post-fertilization or day 6 post-fertilization (after embryo transfer on day 5). As the embryo progresses to the blastocyst stage, both protease and trypsin activity are seen to increase (A: n=49, P=0.006; B: n=38; P=0.0015)

FIG. 3 depicts the selective up-regulation of TMPRSS2 and PRSS8 at the blastocyst stage in media from both human and mouse pre-implantation embryos. (A: n=18, P<0.0001 [TMPRSS2: mouse]; n=20, P<0.0001 [Tmprss2: human]) (C: n=18, P<0.0001 [PRSS8: mouse]; n=20, P<0.0001 [Prss8: human]). B and D depict immunofluorescent labelling of both human and mouse blastocysts using antibodies for the conserved encoded proteins to highlight the presence of TMPRSS2 or PRSS8.

FIG. 4 depicts the levels of trypsin activity (A), and abundance of TMPRSS2 (B) and PRSS8 (C) in drops of embryo-condition media harvested on day 6 post-fertilization (after embryo transfer on day 5). Trypsin activity and TMPRSS2 levels were found to be lower in media from embryos resulting in positive pregnancies (P) compared to the levels in media from embryos resulting in no pregnancy (NP) (n=20, P=0.0015 [Trypsin]; n=19, P=0.0007 [TMPRSS2]). Conversely, PRSS8 levels were higher where embryos resulted in positive pregnancies (P) as compared with embryos that resulted in no pregnancy (NP) (n=32, P=0.0071 [PRSS8]). The Mann Whitney test was used for statistical comparison.

### DETAILED DESCRIPTION

### A. Definitions

Unless defined otherwise, all technical and scientific terms used herein have the same meaning as commonly understood by the ordinary person skilled in the art to which the invention pertains. Without limiting any term, further clarifications of some of the terms used herein are provided below.

As used herein, the term “embryo” refers to the product of fertilization of an oocyte. Embryos assessed using the methods described herein will typically be embryos produced by in vitro fertilization.

As used herein, the term “cleavage-stage” refers to an early stage of embryo development where the cells are undergoing the early rounds of cell division. In particular, post-fertilization the one-cell embryo undergoes a series of cleavage divisions, progressing through a 2-cell, 4-cell, 8-cell and 16-cell stage.

As used herein, the term “blastomeres” is used to refer to the cells within cleavage stage embryos.

As used herein, the term “blastocyst” refers to an embryo at distinct stages of its development. The stages at which an embryo is defined as a blastocyst encompass the embryo from early blastocyst through to late blastocyst formation. The blastocyst consists of two main parts, an area denser with cells called the inner cell mass which will eventually form the foetus, and the trophectoderm which will go on to be the placenta. The stage of embryo development at which an embryo is defined as a blastocyst is typically between 5 and 10 days post-fertilization and/or between the morula stage and before the amniotic sac forms.

As used herein, the term “IVF” or “in vitro fertilization” refers to a method of assisted reproductive technology for the treatment of infertility. IVF is the process of fertilization of an egg (oocyte) with sperm outside of the body of the mother. The fertilised egg, or embryo, is usually cultured in vitro for some period of time or until a desired developmental milestone is reached. For example, some embryos are cultured until the embryo reaches 4-8 cells, or until reaching the blastocyst stage. Other embryos may be cultured for a period of time such as 5 or 6 days post-fertilization, for example. After the desired culturing has been accomplished, embryo selection and embryo transfer occur wherein an embryo is selected and transferred into the uterus of a female recipient. In some cases, selected embryos may not be transferred immediately but may be preserved, for example cryopreserved, for later transfer.

As used herein, the term “embryo transfer” means the process wherein an embryo produced by IVF is transferred to the uterus of a female recipient. As used herein, the term “single embryo transfer” means the transfer of a single embryo into the uterus of a female recipient. This is to be contrasted with the process whereby multiple embryos are transferred into the uterus of a female recipient in order to increase the likelihood of success in establishing a pregnancy.

As used herein, the term “embryo-conditioned medium” refers to medium in which an embryo, typically an embryo produced by IVF, has been grown prior to transfer or preservation. Embryos produced by IVF are typically cultured in single “droplets” or “drops” of medium. These “droplets”, “drops” or “microdroplets” may be, for example, 20 μl or 25 μl of medium.

As used herein, the term “implantation” means the process whereby an embryo adheres to the wall of the uterus; this process is typically required in order for a pregnancy to continue to term. In situations where the embryo is produced by IVF, the success of implantation post embryo transfer is a major factor determining whether or not the female recipient carries the pregnancy to term or whether there is a miscarriage following embryo transfer.

As used herein, the term “viability”, as used in the context of “assessing embryo viability” means the potential for an embryo to implant and give rise to a pregnancy.

As used herein, the term “viable embryo” is used interchangeably with “developmentally-competent embryo” and means an embryo that is capable of successful implantation post embryo transfer and/or an embryo that is capable of successful implantation and that gives rise to a pregnancy resulting in a live birth. Embryo viability is affected by multiple factors including but not limited to the chromosomal complement of the cells of the embryo. As used herein, the term “viable embryo” is intended to cover an embryo that is viable for the reason that it contains a normal chromosome complement and/or does not have any chromosomal abnormalities.

As used herein, the term “non-viable embryo”, used interchangeably with “unviable embryo”, “developmentally-incompetent embryo” and “developmentally-impaired embryo” refers to an embryo that is not capable of successful implantation post embryo transfer and/or an embryo that, post-transfer, is not capable of giving rise to a pregnancy resulting in a live birth. Non-viable embryos may also be defined as embryos that will result in miscarriage post embryo transfer. Embryos may be non-viable for a number of reasons including the presence of chromosomal abnormalities e.g. aneuploid embryos. As used herein, the term “non-viable embryo” encompasses embryos that are non-viable as a result of chromosomal abnormalities.

As used herein, the term “TMPRSS2” refers to a protein belonging to the serine protease family called transmembrane protease, serine 2; it is an enzyme encoded by the TMPRSS2 gene (see NCBI database Gene ID: 7113 for the human homolog). TMPRSS2 is also known as PP9284 and PRSS10.

As used herein, the term “PRSS8” refers to a protein belonging to the serine protease family called protease, serine, 8; it is an enzyme encoded by the PRSS8 gene (see NCBI database Gene ID: 5652 for the human homology). PRSS8 is also known as CAP1 and PROSTASIN.

### B. Methods of Assessing Embryo Viability

In a first aspect, the present invention provides methods of assessing or predicting the viability of an embryo. The embryo is an embryo produced by in vitro fertilization, which is typically being assessed for viability to determine whether the embryo should or should not be transferred to a female recipient, or whether the embryo should or should not be stored (e.g. cryopreserved) for use at a later date.

The methods of the invention are carried out for the purposes of assessing how likely it is, that for any given embryo produced by IVF, the embryo will be viable or non-viable. A viable embryo is defined above as an embryo that will successfully implant following embryo transfer into the uterus and/or give rise to a pregnancy that results in a live birth. A non-viable embryo is defined above as an embryo that will not successfully implant following embryo transfer into the uterus or if implanted, will result in a subsequent miscarriage. It follows that the methods of the present invention are carried out to assess whether an embryo is viable i.e. it will implant and result in a successful pregnancy, and/or whether an embryo is non-viable i.e. following transfer, there will be a miscarriage.

As with other screening methods, the methods of the invention may not be 100% accurate in terms of predicting the viability of each embryo. The methods of the present invention aim to achieve a high degree of sensitivity and/or specificity in predicting whether any given embryo will be viable or non-viable. This is achieved by use of the protein markers described herein. Methods of the present invention may be used to predict whether a given embryo will be viable or non-viable with at least 70%, at least 80%, at least 90%, at least 95% accuracy.

The methods of the invention are based on measuring the level and/or activity of at least one protein marker in a sample of culture medium, wherein the sample has been obtained from the in vitro culture medium of an embryo produced by IVF. Under routine IVF procedures, embryos are typically cultured in individual drops or droplets of culture media, wherein the drop or droplet has a very small volume, typically in the range 20-50 μl. This culture medium is normally discarded after transfer of the embryo and as such, the methods of the invention use a by-product of in vitro embryo culture to assess viability. The methods of the invention are thus advantageous as compared with methods that assess embryo viability directly, for example by sampling of embryonic cells, since they involve no direct manipulation of the embryo.

In certain embodiments, the sample of culture medium analysed according to the present methods will be the complete drop or droplet of embryo-conditioned medium remaining after the embryo has been transferred to the uterus of a female recipient. In certain embodiments, the sample of culture medium analysed will have a volume of 20-50 μl or 20-40 μl or 20-25 μl. The sample of culture medium for analysis will need to be obtained from the in vitro culture medium of an individual embryo i.e. the medium from which the sample is obtained must only have been conditioned by one embryo i.e. the embryo for which the viability is to be assessed.

The method by which the sample of culture medium is obtained should not itself compromise the viability of the embryo. Importantly, the sample of culture medium should be obtained without destruction of the embryo. In certain embodiments, the sample will be obtained by aspiration of some or all of the culture media whilst avoiding damage to the embryo.

In certain embodiments of the present invention, the samples of medium are directly analysed to determine the levels of the specific proteases described herein, wherein direct analysis means that the samples are not processed prior to analysis. In certain embodiments, the samples for analysis according to the present methods may have been obtained from the culture medium of an embryo and subsequently stored prior to analysis, for example stored at −80° C. For embodiments in which the samples are stored or processed prior to analysis, the storage and/or processing should not significantly alter the levels and/or activity of the protein markers in the sample.

The culture medium in which the embryo is cultured post-fertilization and from which the sample for analysis is taken may be any culture medium suitable for the growth of embryos produced by IVF. Culture medium used may be selected from, for example, ISM1 ™ (Origio), BlastAssist™ (Origio), ORIGIO® Sequential Series™, Q1 (Quinn's Protein Plus Cleavage Medium), Q2 (Quinn's Advantage™ Protein Plus Blastocyst Medium) (Sage In Vitro Fertilization), or a combination thereof. In a preferred embodiment, the culture medium used does not already contain the proteins of interest, TMPRSS2 or PRSS8, or contains minimal amounts thereof. Alternatively, or in addition, the culture medium may not contain enzymes, for example proteases or protease inhibitors, that target or alter the levels and/or activity of TMPRSS2 or PRSS8.

The sample for analysis according to the present methods may be obtained from the in vitro culture medium of an embryo when the embryo is at the blastocyst stage, wherein a blastocyst is as defined elsewhere herein. Alternatively, the sample for analysis according to the present methods may be obtained when the embryo is at the cleavage stage, for example the two-cell stage. In certain embodiments, the sample for analysis is taken from the in vitro culture medium of an embryo 2, 3, 4, 5, 6 or 7 days post-fertilization. In certain embodiments the sample for analysis is taken from the in vitro culture medium of an embryo between days 2 and 6 (inclusive) post-fertilization, or between days 3 and 5 (inclusive) post-fertilization. In certain embodiments the embryo has been present in the culture medium for the full duration of time i.e. the same number of days. In certain embodiments, the sample for analysis is taken from the in vitro culture medium of an embryo 4, 5 or 6 days post-fertilization, wherein the culture medium has been changed at least once in the period post-fertilization, for example on day 3. For example, where embryos are transferred on day 5, the media may have been changed on day 3 post-fertilization. In preferred embodiments, the sample for analysis is obtained at day 5 post-fertilization, wherein the embryo has been present in the culture medium for all 5 days. The sample for analysis may be taken on the same day that the embryo is removed from the culture medium or at a time after the embryo has been removed from the culture medium. For example, the embryo may be cultured in the medium for 2 or 5 days post-fertilization and subsequently removed for transfer or storage (e.g. cryopreservation), and the sample may subsequently be obtained 1 day after embryo removal, 2 days after embryo removal, 3 days after embryo removal, up to 5 days after embryo removal, provided that the levels of the protein markers described herein are not significantly changed following removal of the embryo. The point at which the sample is taken for analysis may be dependent upon the day on which embryo transfer to the recipient female occurs. For example, if an embryo is transferred on day 2 post-fertilization, then the sample of culture medium may be obtained on day 2 post-fertilization. Alternatively, if an embryo is transferred on day 2 post-fertilization, then the sample of culture medium may be obtained 5 days after embryo removal. In a preferred embodiment wherein the embryo is transferred to the female recipient on day 5 post-fertilization, the sample is obtained from the culture medium on day 5 post-fertilization. Alternatively, if an embryo is transferred on day 5 post-fertilization, then the sample of culture medium may be obtained 2 days after embryo removal. Alternatively, the embryo may be transferred on day 3 post-fertilization.

The methods of the present invention may be used to assess the viability of an embryo, which is preferably a mammalian embryo. In particularly preferred embodiments-of the invention, the methods are carried out for the purposes of assessing the viability of human embryos produced by IVF.

### C. Measuring Protein Markers: TMPRSS2 and PRSS8

The present invention provides methods wherein samples of media are analysed to measure the level and/or activity of a protein marker selected from TMPRSS2 or PRSS8. As reported herein, the levels of these two markers in embryo-conditioned medium has been found to correlate with embryo viability. In particular, levels of these two markers have been found to correlate with successful implantation. Therefore determining the levels of one or both of these markers in samples of media taken from cultured embryos is an effective means to assess embryo viability.

As reported herein, TMPRSS2 levels were found to be lower in media obtained from embryos that were subsequently successfully implanted as compared with media obtained from embryos that were deemed morphologically unsuitable for transfer, or that did not implant successfully. In contrast, PRSS8 levels were found to be higher in media obtained from embryos that were subsequently successfully implanted as compared with media obtained from embryos that were deemed morphologically unsuitable for transfer, or that did not implant successfully.

The methods of the present invention involve measuring the level and/or activity of a protein marker selected from either TMPRSS2 or PRSS8 in a sample of culture medium obtained according to any of the embodiments described above. In preferred embodiments, the methods involve measuring the level of one or both of the protein markers described herein.

Protein levels may be measured using any of the techniques known to those skilled in the art for qualitatively or quantitatively assessing protein levels including but not limited to Western blotting, protein microarray, ELISA or FACS analysis.

In preferred embodiments, the level of the protein marker is measured by enzyme-linked immunosorbent assay (ELISA). ELISA kits for the detection of TMPRSS2 and PRSS8 are commercially available, and any suitable kit may be used on conjunction with the methods of the present invention. For example, the total level of TMPRSS2 in the samples may be measured using the Human TMPRSS2 ELISA Kit (E-EL-H1418, Elabscience) according to the manufacturer's protocol. The total abundance of PRSS8 in the samples may be measured using RayBio® Human Prostasin ELISA Kit (ELH-Prostasin, RayBiotech, Inc.) according to manufacturer's protocol.

Alternatively or in addition to measuring the levels of one or more of the protein markers, the activity of the protein markers may be measured using any suitable technique known to those skilled in the art. The activity of the protein marker may be determined as an indirect assessment of the levels of that marker in the sample. TMPRSS2 and PRSS8 are both serine proteases, and therefore any technique suitable for the assessment of serine protease activity may be used according to the methods described herein. In embodiments wherein the activity of TMPRSS2 is measured, it would be preferable for the technique or assay used to assess serine protease activity to be specific for the detection of TMPRSS2 activity. Similarly, in embodiments wherein the activity of PRSS8 is measured, it would be preferable for the technique or assay used to assess the serine protease activity to be specific for the detection of PRSS8 activity.

In certain embodiments of the invention, the methods will involve measuring the level and/or activity of TMPRSS2 and PRSS8 in a sample of culture medium obtained from the in vitro culture medium of an embryo, wherein the levels and/or activity of both markers are used in the assessment of embryo viability.

In certain embodiments, the level and/or activity of the protein marker may be used to predict embryo viability by determining the level and/or activity of the protein marker relative to a sample of control unconditioned culture medium, also referred to herein as “control culture medium” or “control”. The control culture medium will typically be the same in composition to the culture medium used to culture the embryo but will be unconditioned i.e. will not have been contacted with an embryo. The protein marker may be entirely absent from the control culture medium such that the level and/or activity of the protein marker is zero in the control. Alternatively, the control culture medium may contain a basal level of protein marker relative to which the level of the protein marker in the sample of conditioned media is determined.

As reported elsewhere herein, the present inventors have found that the levels of the protein marker TMPRSS2 are higher in culture media obtained from the in vitro cultures of embryos that are non-viable as compared with the levels determined in culture media obtained from the in vitro cultures of embryos that are viable. Therefore, in certain embodiments wherein the level and/or activity of the protein marker TMPRSS2 is determined relative to control culture medium, the embryo can be assessed as viable if the level and/or activity of the marker is increased by less than 40% relative to control, preferably less than 30% relative to control, preferably less than 20% relative to control, preferably less than 10% relative to control. In certain embodiments, the embryo can be assessed as viable if the level and/or activity of the marker is equivalent or decreased relative to control. Conversely, the embryo can be assessed as non-viable if the level and/or activity of the TMPRSS2 marker is increased by more than 10% relative to control, preferably increased by more than 20% relative to control, preferably increased by more than 30% relative to control, preferably increased by more than 40% relative to control.

As reported elsewhere herein, the present inventors have found that the levels of the protein marker PRSS8 are higher in culture media obtained from cultures of embryos that are viable as compared with the levels determined in culture media obtained from cultures of embryos that are non-viable. Therefore, in certain embodiments wherein the level and/or activity of the protein marker PRSS8 is determined relative to control culture medium, the embryo can be assessed as viable if the level and/or activity of the marker is increased relative to control, preferably increased by at least 10%, preferably increased by at least 20%, preferably increased by at least 30%, preferably increased by at least 40% relative to control. Conversely, the embryo can be assessed as non-viable if the level and/or activity of the PRSS8 marker is equivalent to or decreased relative to control or increased by less than 40% relative to control, preferably less than 30% relative to control, preferably less than 20% relative to control, preferably less than 10% relative to control.

In certain embodiments, the methods of the invention include an additional step wherein the level and/or activity of either TMPRSS2 or PRSS8 is compared with a reference value for said protein marker. This comparison step allows one to use the information obtained in the first step of the method, i.e. wherein the level or activity of the protein marker is determined in a sample of embryo-conditioned media, to assess the viability of the embryo that was cultured in the media from which the sample was obtained.

The reference value for each protein marker may itself be indicative of a viable embryo or a non-viable embryo. It follows, that the information that can be determined from a comparison of the sample results with the reference value will depend on the nature of the reference value.

In certain embodiments, the protein marker is TMPRSS2 and the reference value for this marker is indicative of a viable embryo. In such embodiments, if the level and/or activity of TMPRSS2 is equal to or lower than the reference value, then the embryo is assessed as viable. Alternatively or in addition, if the level and/or activity of TMPRSS2 is higher than the reference value, then the embryo may be assessed as non-viable or unviable. In certain embodiments, the protein marker is TMPRSS2 and the reference value for this marker is indicative of a non-viable embryo. In such embodiments, if the level and/or activity of TMPRSS2 is equal to or lower than the reference value, then the embryo is assessed as non-viable. Alternatively or in addition, if the level and/or activity of TMPRSS2 is higher than the reference value, then the embryo may be assessed as viable.

In certain embodiments, the protein marker is PRSS8 and the reference value for this marker is indicative of a viable embryo. In such embodiments, if the level and/or activity of PRSS8 is equal to or higher than the reference value, then the embryo is assessed as viable. Alternatively or in addition, if the level and/or activity of PRSS8 is lower than the reference value, then the embryo may be assessed as non-viable or unviable. In certain embodiments, the protein marker is PRSS8 and the reference value for this marker is indicative of a non-viable embryo. In such embodiments, if the level and/or activity of PRSS8 is equal to or lower than the reference value, then the embryo is assessed as non-viable. Alternatively or in addition, if the level and/or activity of PRSS8 is higher than the reference value, then the embryo may be assessed as viable.

For embodiments in which a comparison is made with a reference value, the reference value for the protein marker may have been pre-determined. It would be within the capabilities of the skilled person to generate a comparator “reference value” based on the analysis of multiple samples, and compare the samples under test against this comparator. For example, reference values indicative of viable and non-viable embryos may be obtained or have been obtained previously by measuring the level and/or activity of one of the protein markers described herein in multiple samples, each sample obtained from the in vitro culture medium of a separate embryo produced by in vitro fertilization. The fate of the embryos from which the samples derive could be monitored and each embryo could be classified as viable or non-viable dependent on the outcome post-transfer. The results obtained from the samples taken from the viable embryos could be combined, for example averaged, to provide a reference value indicative of a viable embryo for a given protein marker. Similarly, the results obtained from the samples taken from the non-viable embryos could be combined, for example averaged, to provide a reference value indicative of a viable embryo for a given protein marker. The reference value may be calculated based on data obtained from the analysis of at least 20 samples, at least 50 samples, at least 100 samples, at least 500 samples, at least 1000 samples. The technique or assay used to analyse each of the samples to determine a reference value may require calibration to correct for run-to-run variation. The results for each sample may also need to be adjusted to take account of differences in culture media, or other parameters that could affect the results obtained.

In certain embodiments, the reference value used for comparison may be determined based on concurrent or previous analysis of one or more samples obtained from the culture medium of embryos produced by in vitro fertilization using oocytes from the same female donor. Alternatively or in addition, the embryos may have been produced by IVF using sperm from the same male donor. For example, if a couple have had a previous attempt at IVF and the levels and/or activity of one or more of the markers described herein were determined for the culture medium from which the one or more previous embryos were transferred, these values may serve as reference values for subsequent IVF treatments. In certain embodiments, the previous IVF treatment(s) may have failed, in which case the reference value will be indicative of a non-viable embryo. In certain embodiments, the previous IVF treatment(s) may have been successful, in which case the reference value will be indicative of a viable embryo.

In further embodiments, the methods include a further step of selecting a viable embryo, preferably for single embryo transfer, or for multiple embryo transfer, or for storage such as cryopreservation of spare viable embryos.

### D. Methods of Selecting Embryos for Implantation

In a further aspect, the present invention is directed to methods for selecting embryos suitable for implantation based on a comparison of samples derived from the in vitro culture media of embryos produced by in vitro fertilization using oocytes from the same female donor. All relevant embodiments of the invention described above in connection with the first aspect of the invention are equally applicable to this second aspect of the invention, and can be combined where appropriate.

In this second aspect of the invention, the level and/or activity of a protein marker selected from either TMPRSS2 or PRSS8 is measured in two or more samples of culture medium, wherein the samples have been obtained from the in vitro culture media of embryos produced by in vitro fertilization using oocytes from the same female donor. In certain embodiments, the embryos from which the samples are taken for assessment have been produced by IVF using oocytes and sperm from the same female and male donors, respectively. The measurements determined for each of the samples tested are compared in order to select an embryo suitable for implantation. As used herein, the term “selecting an embryo suitable for implantation” should be taken to mean that the embryo is selected for embryo transfer. The embryo may still fail to implant when transferred to the uterus of a female recipient but is still selected as “suitable for implantation” based on a comparison of two or more samples according to the methods described herein.

The methods may be carried out using three or more, four or more, five or more, six or more samples wherein each sample has been obtained from the culture medium of an embryo produced by in vitro fertilization and each embryo has been produced using an oocyte from the same female donor, optionally wherein the embryo has been produced using sperm from the same male donor. In certain embodiments, the methods described herein will be carried out using samples obtained from the in vitro culture medium of embryos produced using oocytes from the same female donor and sperm from the same male donor.

For embodiments wherein the protein marker is TMPRSS2, the methods involve selecting the embryo having the lowest TMPRSS2 level and/or activity. For embodiments wherein the protein marker is PRSS8, the methods involve selecting the embryo having the highest PRSS8 level and/or activity.

In certain embodiments, the methods of selecting an embryo for implantation according to this second aspect of the invention may involve one or more additional steps wherein the viability of the one or more embryos is independently assessed according to steps described above in connection with the first aspect of the invention. For example, the methods may involve one or more additional steps to independently assessing the viability of one or more of the embryos from which the media samples are obtained, by measuring the level and/or activity of a protein marker selected from TMPRSS2 and PRSS8, and comparing this level and/or activity to a sample of control unconditioned culture medium. As explained above in connection with the methods of the first aspect of the invention, this comparison with control unconditioned culture medium can be used to assess the viability of an embryo. Alternatively, the methods of the second aspect of the invention may involve one or more additional steps to independently asses the viability of one or more of the embryos from which the media samples are obtained, by measuring the level and/or activity of a protein marker selected from TMPRSS2 and PRSS8, and comparing this with a reference value wherein the reference value is indicative of a viable or non-viable embryo.

For embodiments of the methods described herein involving one or more additional steps to independently assess the embryo viability, the assessment of embryo viability may be performed exclusively on the embryo selected for implantation i.e. the embryo identified as having the lowest TMPRSS2 levels and/or the embryo identified as having the highest PRSS8 levels.

The embryo(s) selected for implantation according to the methods described herein may be transferred to a female recipient, preferably in a process of single embryo transfer, or may be stored, for example cryopreserved, for transfer at a later time.

E. Combining the Methods with other Methods for Pre-Implantation Screening

The methods as described herein may be combined with alternative methods for pre-implantation embryo screening, for example to further improve the sensitivity and/or specificity of the assessment. Therefore, the levels and/or activity of TMPRSS2 and/or PRSS8 in the media may be used as a marker of embryo viability, optionally in combination with any of the existing techniques described herein for the assessment of embryo viability.

In certain embodiments, the methods of the invention may be combined with morphological analysis of the embryo. Morphological analysis may be performed on an embryo any time from day 2 to day 6 post-fertilization, and the conditioned media sample may be obtained for analysis according to the methods described herein after the morphological analysis. Alternatively, the conditioned media sample may be obtained for analysis according to the methods described herein prior to the morphological analysis. Embryos displaying the desired characteristics after both morphological analysis and protein marker analysis of TMPRSS2 and/or PRSS8 may be selected for embryo transfer or cryopreservation where appropriate.

The methods of the invention may be used in combination with other non-invasive techniques or invasive techniques such as PGS. This would allow for the assessment of both genetic chromosomal abnormalities by PGS in combination with analysis of protein markers of implantation potential using the methods of the present invention. Embryos displaying the desired characteristics after both PGS and protein marker analysis of TMPRSS2 and/or PRSS8 may be selected for embryo transfer or cryopreservation, where appropriate.

In certain embodiments, the samples analysed according to the methods described herein may be obtained from the in vitro culture medium of embryos pre-selected by an independent assessment method. For example, embryos determined to be viable by morphological analysis may be analysed according to the methods described herein to assess whether the embryo is suitable for embryo transfer or storage for potential transfer at a later stage.

The invention will now be further understood with reference to the following non-limiting examples.

### EXAMPLES

### Example 1

### Analysis of Human and Murine Embryonic Transcriptome for Serine Protease Genes Expressed Throughout Pre-Implantation Development

In silico analysis was performed on publicly available datasets from the Gene Expression Omnibus project, the GEO project, (Edgar et al. 2002 Nucleic Acids Research, 30:1, 207-210), accession number GSD3959 (homo sapiens) and GDS813 (mus musculus). Statistical analysis of the datasets obtained from the GEO project was performed using SPSS Software, IBM.

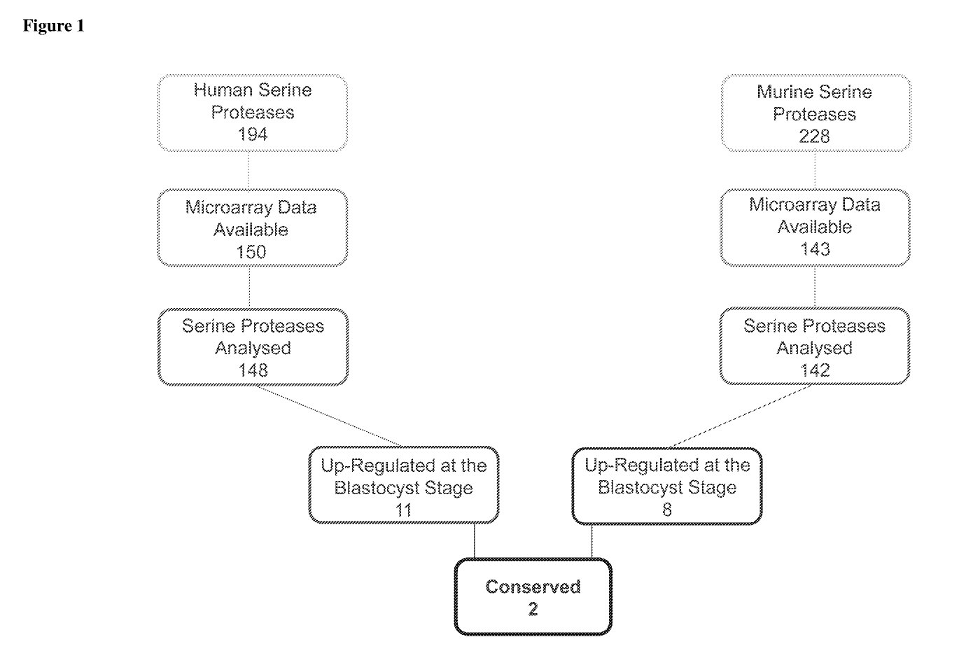

As shown in FIG. 1, a total of 194 human and 228 murine serine protease genes were identified in the available datasets, and microarray data on expression during pre-implantation embryo development was available for 150 and 143 of these genes, respectively. 11 human serine proteases and 8 mouse serine proteases were identified as being selectively up-regulated at the blastocyst stage and 2 of these proteases were found to be conserved between species. These two serine proteases are TMPRSS2 and PRSS8.

### Example 2

### Measurement of Protease and Trypsin Activity in Embryo-Conditioned Medium (ECM)

Experiments were carried out to assess total protease and trypsin activity in samples of conditioned media obtained from the in vitro culture of embryos i.e embryo-conditioned medium.

Embryo conditioned medium was obtained from two IVF units: Centre for Reproductive Medicine (CRG), Universitair Ziekenhuis Brussel, Laarbeeklaan 101, 1090 Brussels, Belgium; Centre for Reproductive Medicine (CRM), University Hospitals Coventry & Warwickshire, Clifford Bridge Road, Coventry CV2 2DX, UK.

Embryos produced were cultured in individual 20 μl drops of ISM1™ (Origio) and BlastAssist™ (Origio). During this study the CRG changed their culture method to include the ORIGIO® Sequential Series™ (Fert™, Cleav™, Blast™).

Embryos from CRG Brussels were cultured individually in 25 μl drops of Q1 (Quinn's Protein Plus Cleavage Medium) and Q2 (Quinn's Advantage™ Protein Plus Blastocyst Medium) (Sage In Vitro Fertilization). During this study the CRG changed their culture method to include the ORIGIO® Sequential Series™ (Fert™, Cleav™, Blast™).

At the IVF units, samples or “drops” of media were collected either at day 2 or day 6 post-fertilization. For embryos that were suitable for culture to the blastocyst stage, samples or “drops” of culture media were collected on day 6 post-fertilization following transfer of the embryo to the uterus of a recipient female on day 5 post-fertilization or cryopreservation of the embryo, where appropriate. For embryos that were transferred to a female recipient on day 2, samples of media were collected at the time of transfer. “Empty drops” i.e. samples of culture media that had not been in contact with any embryo were used as internal controls. All samples (drops) were stored at −80° C. and thawed on the day of use for use in the following assays:

Protease activity was measured using the EnzChek Protease Assay Kit for green fluorescence (E6638, Life Technologies) according to the manufacturer's protocol. This kit contains a casein derivative that has been extensively labelled with the pH-insensitive, green-fluorescent BODIPY® FL dye, which results in a quenching of the fluorescent dye. Protease-catalyzed hydrolysis releases the highly-fluorescent BODIPY® FL dye-labelled peptides, allowing for quantitative detection of protease activity in solution. The green-fluorescent BODIPY® FL dye has excitation and emission spectra similar to those of fluorescein. Samples were thawed, warmed to room temperature and diluted as follows; 7.5 μl embryo-conditioned media+92.5 μl 1× Digestion Buffer per well. Samples and standards were applied in duplicate and incubated for 24 hours at room temperature, protected from light. Fluorescence was measured at 505/513 nm, and concentrations determined by comparison to the standard curve.

83 embryo drops were analysed: 17 collected from day 2 embryos and 66 from day 5 embryos. 100% of drops fell within the standard curve and were suitable for analysis.

The Trypsin Activity Assay Kit (Colorimetric) (ab10253, ABCAM) was used according to the manufacturer's instructions to accurately measure trypsin activity in ECM. The cleavage of a given substrate, by trypsin, generates p-nitroaniline (p-NA), which is detected at λ=405 nm and the resulting colour intensity is thus proportional to p-NA content.

70 embryo drops were analysed, 20 collected from day 2 embryos and 50 from day 5 embryos. 97% of drops fell within the standard curve and were suitable for analysis.

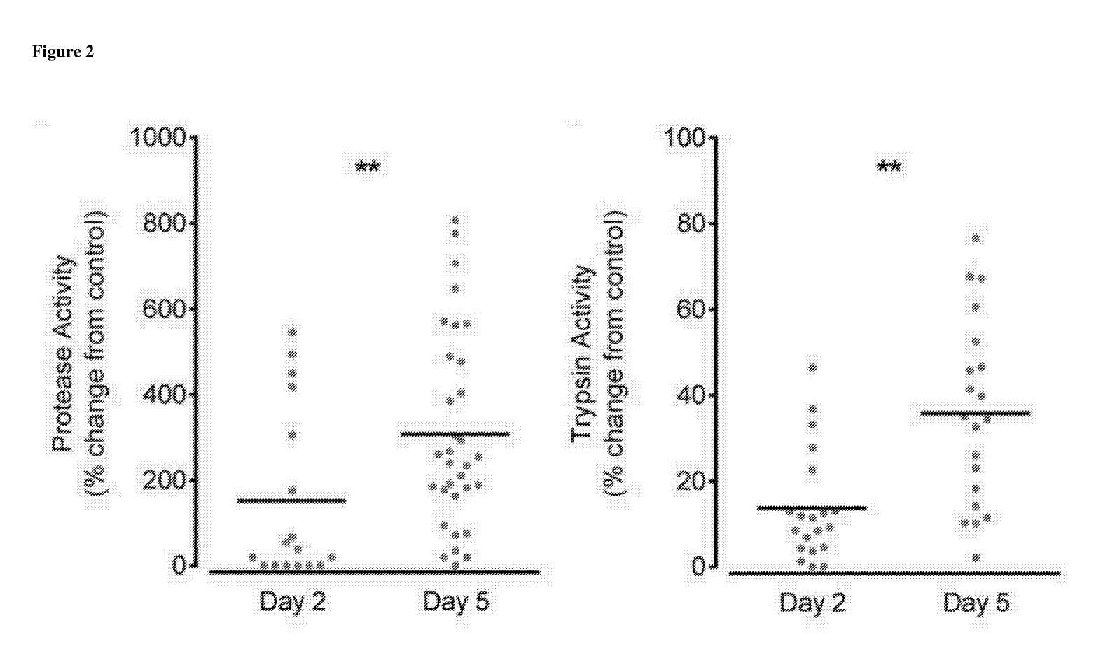

The results are shown in FIG. 2. Protease activity and trypsin activity increased with development to the blastocyst stage, and were detectable in the embryo-conditioned media. Higher activity levels were observed in media from day 5 embryos compared to media from day 2 embryos.

### Example 3

### Immunofluorescent Labelling of TMPRSS2 and PRSS8 in both Human and Mouse Blastocyts

Human and mouse embryos were collected at various stages of development, fixed in 4% Para-formaldehyde/PBS for 1 hour at room temperature then stored at 2-8° C.

Embryos selected for use were washed in PBS, permeabilised for 1 hour in 0.01% Triton X-100/PBS at room temperature and incubated in BSA for 1 hour at 4° C. to prevent non-specific binding. Embryos were then incubated in selected primary antibodies (diluted 1:100 in PBS) or BSA in the case of the negative control, overnight at 4° C. The antibodies used in this study are shown below in Table 1.

TABLE 1

| Name | Company | Cat. Number |
|---|---|---|
| Alpha-1-Microglobulin | Novus Biologicals | H00000259-M01 |
| Enterokinase* | Novus Biologicals | NBP1-55616 |
| NANOG* | Fisher Scientific | 12907229 |
| PRSS8 (Protease. serine, 8)* | Antibodies Online | ABIN761891 |
| Anti-SPINT1* | Sigma | SAB1409704-50UG |
| TMPRSS2/PRSS10* | Antibodies Online | ABIN716876 |

*No commercially available blocking peptides

AMBP blocking peptide (Fitzgerald, 33R-9450-FIT)

The following day, embryos were washed in BSA for 10 minutes at 4° C. and then incubated in the secondary antibodies (diluted 1:100 in PBS) for 2 hours at 4° C. Embryos were then washed in BSA and mounted onto 35 mm glass-bottomed petri dishes (MatTek, US) in 10 μ1 Vectashield® With DAPI (H-1200, Vector) and overlaid with a glass coverslip. Embryos were visualised using a confocal microscope (LSM 510 META, Carl Zeiss, UK). Total number of oocytes/embryos stained for immunocytochemistry:

TABLE 2

| Origin | Mouse | Human |
|---|---|---|
| ENTK | 8 | 21 |
| AMBP | 8 | 24 |
| PRSS8 | 9 | 19 |
| TMPRSS2 | 9 | 11 |
| SPINT1 | 4 | 5 |
| NANOG | 4 | 3 |
| Total | 42 | 83 |

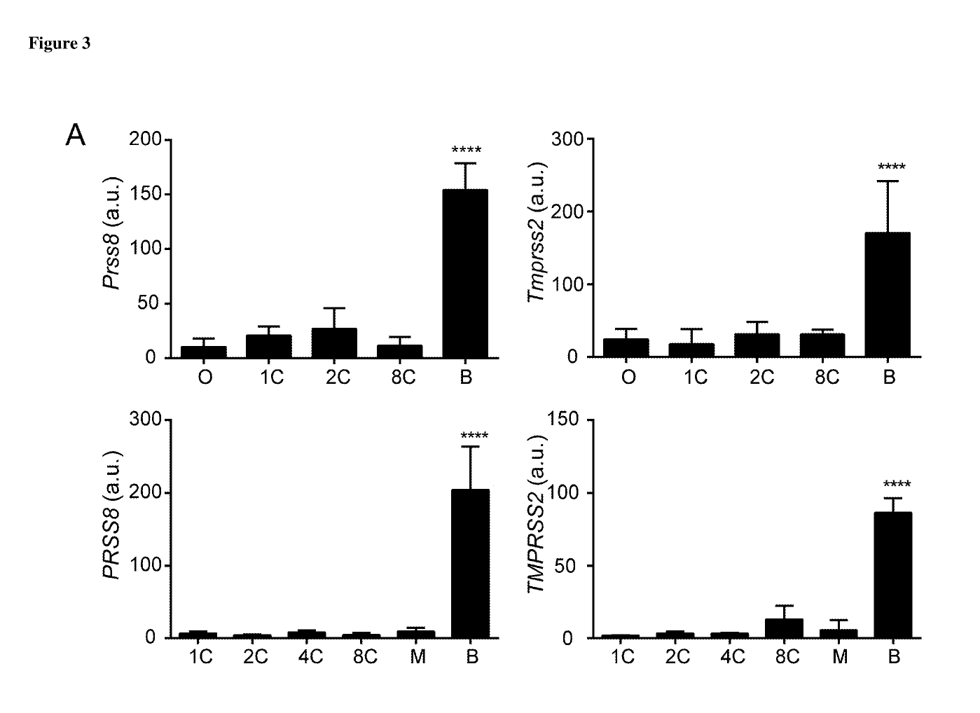

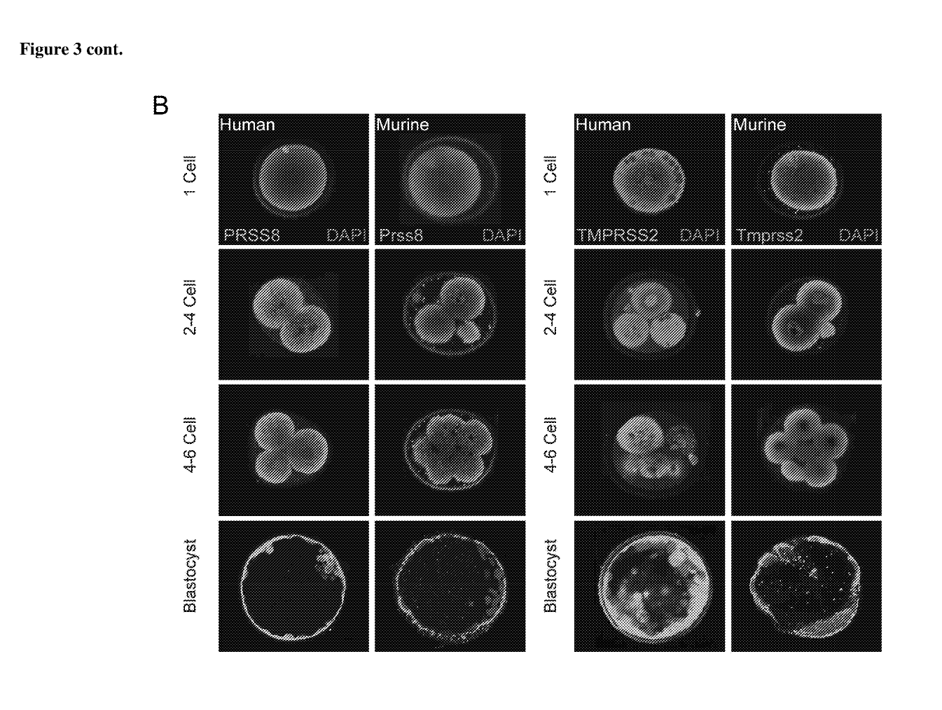

The results of the immunofluorescent staining experiments are shown in FIG. 3. In both mouse and human blastocysts, both TMPRSS2 and PRSS8 protein levels were selectively upregulated at the blastocyst stage during pre-implantation embryo development.

### Example 4

### Measurement of TMPRSS2 Levels and PRSS8 Levels in Embryo-Conditioned Media from Viable and Non-Viable Embryos

Samples or “drops” of media were obtained from embryos cultured and implanted at two IVF clinics, as described in Example 2. These samples were stored at −80° C. and thawed on the day of use for analysis of TMPRSS2 levels and PRSS8 levels by ELISA, as described below.

For the assessment of TMPRSS2 levels, 105 embryo drops were analysed all collected from day 5 embryos. 18% of drops fell within the standard curve for the ELISA and were suitable for analysis.

The total level of TMPRSS2 in the samples was determined using the Human TMPRSS2 ELISA Kit (E-EL-H1418, Elabscience) according to manufacturer's protocol. Samples were thawed, warmed to room temperature and diluted as follows; 7.5ul ECM+92.5 ul Reference Standard & Sample Diluent per well. The samples and standards were loaded, in duplicate, into the 96 well plate, pre-coated with an antibody specific to TMPRSS2. Next, Avidin conjugated to Horseradish Peroxidase (HRP) was added to each well and incubated. After the addition of TMB substrate solution only those wells that contain TMPRSS2, biotin-conjugated antibody and enzyme-conjugated Avidin will exhibit a colour change. The enzyme-substrate reaction was terminated by the addition of sulphuric acid solution and the colour change was measured at 450 nm. The concentration of TMPRSS2 in the samples was then determined by comparing their O.D. to the standard curve.

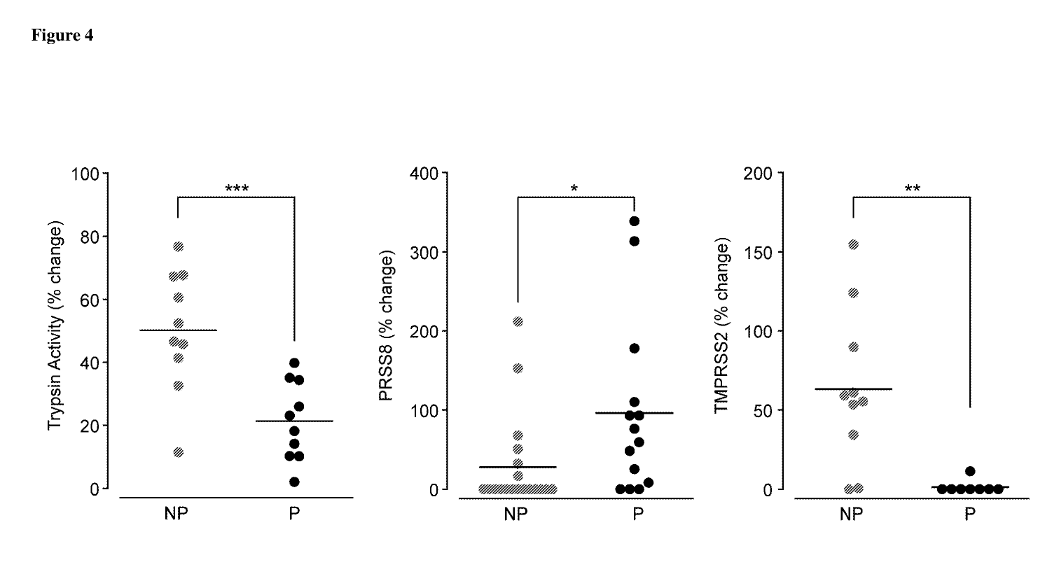

Absolute levels of TMPRSS2 were found to be lower in culture media from embryos that successfully implanted as compared with culture media obtained from embryos that were deemed morphologically unsuitable for transfer, or that did not implant successfully (see FIG. 4B)

For the assessment of PRSS8 levels, 153 embryo drops were analysed, 10 collected from day 2 embryos and 143 from day 5 embryos. 42% of drops fell within the standard curve of the ELISA and were suitable for analysis.

The total abundance of PRSS8 in ECM samples was measured using RayBio® Human Prostasin ELISA Kit (ELH-Prostasin, RayBiotech, Inc.) according to manufacturer's protocol. Samples were thawed, warmed to room temperature and diluted as follows; 7.5 μl ECM+92.5 μl Assay Diluent D per well. Samples and standards were applied to the 96-well plate (mounted with immobilized antibody) in duplicate and incubated overnight at 4° C., with gentle shaking. Following incubation with HRP-conjugated streptavidin and the addition of a TMB substrate solution, the kit provides a colour change from blue to yellow according to the quantity of prostasin present in the sample. The absorbance was read at 450 nm and the sample concentrations were determined by comparison to the standard curve.

PRSS8 levels were found to be higher in culture media from successfully implanted embryos as compared with culture media obtained from embryos that were deemed morphologically unsuitable for transfer, or that did not implant successfully (see FIG. 4C).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

1. A method of assessing the viability of an embryo, wherein the method comprises a step of:
measuring the level and/or activity of a protein marker selected from either TMPRSS2 or PRSS8 in a sample of culture medium, wherein the sample has been obtained from the in vitro culture medium of an embryo produced by in vitro fertilization,
wherein the level and/or activity of the protein marker is indicative of the viability of the embryo.

2. The method of claim 1, wherein the protein marker is TMPRSS2 and the level and/or activity of TMPRSS2 is determined relative to a sample of control unconditioned culture medium, such that if the level and/or activity of TMPRSS2 is increased by less than 40% relative to control, the embryo is assessed as viable and if the level and/or activity of TMPRSS2 is increased by more than 40% relative to control, the embryo is not viable.

3. The method of claim 1, wherein the protein marker is PRSS8 and the level and/or activity of PRSS2 is determined relative to a sample of control unconditioned culture medium, such that if the level and/or activity of PRSS8 is increased relative to control, the embryo is assessed as viable, and if the level and/or activity of PRSS8 is equivalent to or decreased relative to control, the embryo is not viable.

4. The method of claim 1, wherein the method comprises an additional step of:
comparing the level and/or activity of the protein marker measured in the sample with a reference value for said protein marker,
wherein the level and/or activity of the protein marker in the sample relative to the reference value is indicative of the viability of the embryo.

5. The method of claim 4, wherein the protein marker is TMPRSS2 and (a) the reference value for TMPRSS2 is indicative of a viable embryo, such that if the level and/or activity of TMPRSS2 is equal to or lower than the reference value the embryo is assessed as viable, and if the level and/or activity of TMPRSS2 is higher than the reference value the embryo is assessed as not viable; or (b) the reference value for TMPRSS2 is indictive of a non-viable embryo, such that if the level and/or activity of TMPRSS2 is lower than the reference value the embryo is assessed as viable, and if the level and/or activity of TMPRSS is equal to or higher than the reference value the embryo is assessed as not viable.

6. (canceled)

7. The method of claim 4 wherein the protein marker is PRSS8 and (a) the reference value for PRSS2 is indicative of a viable embryo, such that if the level and/or activity of PRSS8 is equal to or higher than the reference value the embryo is assessed as viable, and if the level and/or activity of PRSS8 is lower than the reference value the embryo is assessed as not viable; or (b) the reference value for PRSS8 is indictive of a non-viable embryo, such that if the level of PRSS8 is higher than the reference value the embryo is assessed as viable, and if the level of PRSS8 is equal to or lower than the reference value the embryo is assessed as not viable.

8. (canceled)

9. The method of claim 1, wherein the sample of culture medium has been obtained when the embryo is at the blastocyst stage; or the sample of culture medium has been obtained on day 2, 3, 4, 5 or 6 post-fertilization.

10. (canceled)

11. The method of claim 1, wherein the in vitro culture medium of the embryo does not contain proteases that target TMPRSS2 or PRSS8.

12. The method of claim 1, wherein the embryo is a human embryo.

13. The method of claim 1, wherein the level of the protein marker is measured by ELISA.

14. The method of claim 1, wherein the method comprises a further step of selecting a viable embryo for single embryo transfer.

15. A method of selecting an embryo for implantation, the method comprising the steps of:
(i) measuring the level and/or activity of a protein marker selected from either TMPRSS2 or PRSS8 in two or more samples of culture medium, wherein the samples have been obtained from the in vitro culture media of embryos produced by in vitro fertilization using oocytes from the same female donor,
(ii) comparing the level and/or activity of the protein marker between the two or more samples of (i),
wherein if the marker is TMPRSS2, the embryo from which the sample of culture medium having the lowest TMPRSS2 level and/or activity is obtained is selected for implantation,
or, if the marker is PRSS8, the embryo from which the sample of culture medium having the highest PRSS8 level and/or activity is obtained is selected for implantation.

16. The method of claim 15, wherein in step (i), the samples have been obtained from the in vitro culture media of embryos produced by in vitro fertilization using sperm from the same male donor.

17. The method of claim 15, wherein, for at least one of the samples, the level of the protein marker is determined relative to a sample of control unconditioned culture medium to assess the viability of the embryo,
wherein if the protein marker is TMPRSS2 and the level and/or activity of TMPRSS2 is increased by less than 40% relative to control, the embryo is assessed as viable, or
wherein if the protein marker is PRSS8 and the level and/or activity of PRSS8 is increased relative to control, the embryo is assessed as viable.

18. The method of claim 15, wherein the two or more samples of culture medium have been obtained when the embryos are at the blastocyst stage; or the two or more samples of culture medium have been obtained on day 2, 3, 4, 5 or 6 post-fertilization.

19. (canceled)

20. The method of claim 15, wherein the in vitro culture medium of the embryos does not contain proteases that target TMPRSS2 or PRSS8.

21. The method of claim 15, wherein the embryos are human embryos.

22. The method of claim 15, wherein the level of the protein marker is measured by ELISA.

23. The method of claim 15, wherein the embryo selected for implantation is selected for single embryo transfer.

24. (canceled)

25. (canceled)

26. A method of assessing the viability of an embryo, wherein the method comprises a step of:
measuring the level and/or activity of the protein markers TMPRSS2 and PRSS8 in a sample of culture medium, wherein the sample has been obtained from the in vitro culture medium of an embryo produced by in vitro fertilization,
wherein the level and/or activity of the protein markers is indicative of the viability of the embryo.
